(19) United States
(12) Patent Application Publication
(10) Pub. No.: US 2007/0038033 A1
(43) Pub. Date: Feb. 15, 2007

(54) Cassette based surgical retractor

(75) Inventors: Bryan S. Jones, West Roxbury, MA (US); Timothy Beardsley, Kingston, MA (US); Nick Pavento, Walpole, MA (US); Robert Andrew Howard, Palo Alto, CA (US); Arthur G. Sandoval, San Francisco, CA (US); Joel Bernard Jacobs, San Francisco, CA (US); Daniel Francis Kennedy, San Francisco, CA (US); Daniel Mark Senatore, San Francisco,, CA (US); Jonathan Paul Downing, Foster City, CA (US); Matthew Wallace Peterson, San Francisco, CA (US)

Correspondence Address: LAHIVE & COCKFIELD, LLP, ONE POST OFFICE SQUARE, BOSTON, MA, 02109-2127, US

(73) Assignee: DePuy Spine, Inc., Raynham, MA (US)

(21) Appl. No.: 11/411,002

(22) Filed: Apr. 25, 2006

Related U.S. Application Data
(60) Provisional application No. 60/674,640, filed on Apr. 25, 2005.

(51) Int. Cl. A61B 1/32

(52) U.S. Cl. 600/219; 600/233

(57) ABSTRACT

The present invention provides a retractor featuring cassette assemblies that provide the blades for the retractor and include mechanisms for controlling the angulation and retraction of the blades. The use of such cassette assemblies allow for the addition or removal of blades on the retractor by attaching or detaching the cassette assemblies to a frame. The mechanisms for controlling angulation and retraction of the blades allow a surgeon greater flexibility for creating a surgical site.

### REFERENCE TO RELATED APPLICATION

The present application claims priority to U.S. Provisional Patent Application No. 60/674,640, filed Apr. 25, 2005, entitled Surgical Retractor and incorporated herein by reference.

### FIELD OF THE INVENTION

The present invention relates to a device and method for accessing a surgical site during surgery, such as spinal surgery. More particularly, the present invention relates to a device and method for retracting tissue surrounding a surgical site during a surgery.

### BACKGROUND OF THE INVENTION

In surgical procedures, it is important to minimize trauma to the patient and damage to the tissue as much as possible. To achieve this result surgeons try to keep incisions as small as possible when performing surgical procedures. However, it is necessary that the surgeon performing the delicate surgery have a clear view of the operating field. A variety of retractors are available to keep an incision open and provide a clear field of view of the operation. The retractor is inserted in the incision to hold organs, muscles, arteries and other tissue out of the way and provide a clear view of the spinal region being operated on. The retractor displaces only a small volume when inserted in the incision before it is opened, or “spread” to provide a clear view of the operating field.

Traditional retractors typically have a fixed number of blades with limited ability to position the blades on the retractor. The fixed nature of current retractors requires that a number of different retractors must be made available for a surgical procedure depending on the type and needs of particular surgical procedure.

A need exists for new surgical retractors that provide the surgeon with a greater flexibility so as to be adaptable depending on the needs of a particular surgical procedure to explore and create an operating field and perform surgical procedures in a minimally invasive manner.

### SUMMARY OF THE INVENTION

The present invention provides a retractor featuring cassette assemblies that provide the blades for the retractor and include mechanisms for controlling the angulation and retraction of the blades. The use of such cassette assemblies allow for the addition or removal of blades on the retractor by attaching or detaching the cassette assemblies to a frame.

In a first aspect, a cassette assembly is provided for use in a surgical retractor, the cassette assembly includes a cassette housing configured to attach to a frame; a blade pivotably connected to the cassette housing; a first mechanism configured to control the pivot of the blade; and a second mechanism configured to control retraction of the blade.

In another aspect, a surgical retractor is provided. The surgical retractor includes a frame and a cassette assembly attached to the frame. The cassette assembly includes a cassette housing configured to attach to a frame, a blade pivotably connected to the cassette housing, a first mechanism configured to control the pivot of the blade, and a second mechanism configured to control retraction of the blade.

In another aspect, a method of forming a surgical site in a patient is provided. The method involves creating an incision in the patient, inserting a retractor into the incision in the patient, and retracting the tissue of the patient at the incision with the retractor to form a surgical site. The retractor includes a frame and a cassette assembly attached to the frame. The cassette assembly includes a cassette housing configured to attach to a frame, a blade pivotably connected to the cassette housing, a first mechanism configured to control the pivot of the blade, and a second mechanism configured to control retraction of the blade.

### BRIEF DESCRIPTION OF THE FIGURES

The foregoing and other objects, features and advantages of the invention will be apparent from the following description and apparent from the accompanying drawings, in which like reference characters refer to the same parts throughout the different views. The drawings illustrate principles of the invention and, although not to scale, show relative dimensions

FIG. 1 illustrates an embodiment of a surgical retractor of an illustrative embodiment of the invention.

FIGS. 2A-C illustrate an embodiments of a surgical retractor including a plurality of cassette assemblies according to an illustrative embodiment of the invention.

FIG. 3 illustrates an embodiment of a cassette assembly of the present invention.

FIGS. 4A-B illustrate one embodiment of a clamping mechanism for attaching a cassette assembly to a frame according to an illustrative embodiment of the invention.

FIGS. 4C-E illustrate another embodiment of a clamping mechanism for attaching a cassette assembly to a frame according to an illustrative embodiment of the invention.

FIG. 5 illustrates an embodiment of a retractor of the present invention according to an illustrative embodiment of the invention.

FIG. 6 illustrates one embodiment of a first mechanism for controlling the pivot of the blade of a cassette assembly according to an illustrative embodiment of the invention.

FIGS. 7A-E illustrate another embodiment of a first mechanism for controlling the pivot of the blade of a cassette assembly according to an illustrative embodiment of the invention.

FIG. 8 illustrates an embodiment of a second mechanism for controlling the retraction of the blade of a cassette assembly according to an illustrative embodiment of the invention.

FIG. 9 illustrates an embodiment of a method for creating a surgical site according to an illustrative embodiment of the invention.

FIGS. 10A-C illustrate embodiments of techniques for inserting a retractor according to an illustrative embodiment of the invention.

FIG. 11 illustrates an embodiment of a retractor having the blades retracted according to an illustrative embodiment of the invention.

FIGS. 12A-B illustrate an embodiment of a retractor having the blades angled according to an illustrative embodiment of the invention.

### DETAILED DESCRIPTION OF THE INVENTION

The present invention provides an improved surgical device and method for retracting tissue from an incision and providing access to a surgical site in a patient. The present invention will be described below relative to certain exemplary embodiments in spinal surgery to provide an overall understanding of the principles of the structure, function, manufacture, and use of the instruments disclosed herein. Those skilled in the art will appreciate that the present invention may be implemented in a number of different applications and embodiments and is not specifically limited in its application to the particular embodiments depicted herein. For example, while the illustrative embodiment of the invention relates to a spinal retractor used in spinal surgery, the surgical retractor may be used in any surgical process where access to a surgical site is required.

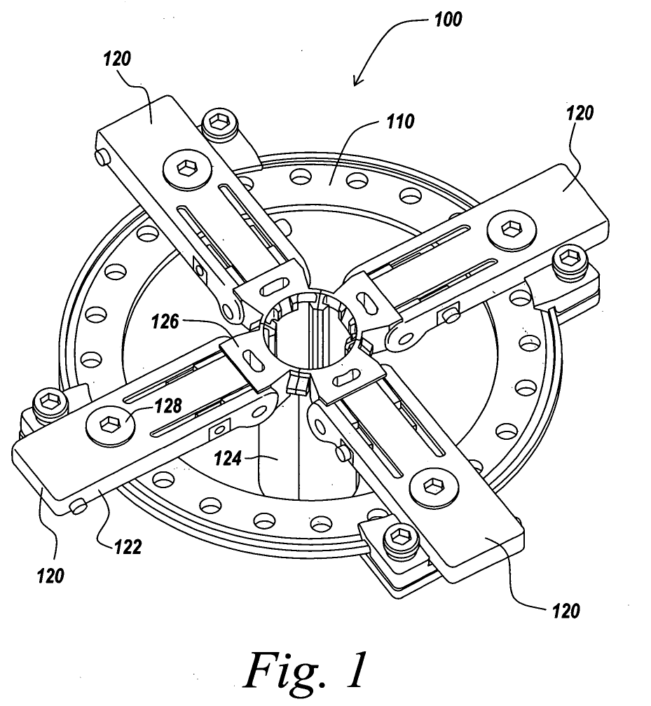

FIG. 1 illustrates an embodiment of a surgical retractor 100 of an illustrative embodiment of the invention. The surgical retractor 100 comprises a frame 110; and a cassette assembly 120 attached to the frame. The cassette assembly includes a cassette housing 122 configured to attach to the frame 110; a blade 124 pivotably connected to the cassette housing 122; a first mechanism 126 configured to control the pivot of the blade 124; and a second mechanism 128 configured to control retraction of the blade 124.

The frame 110 provides a structure for attaching cassette assemblies 120 to form the retractor. In certain embodiments, such as shown in FIG. 1, the frame 110 is substantially circular. However, in other embodiments the frame 110 may be elliptical, oval, rectangular, or other polygonal shape. In still other embodiments, the frame 110 may be linear in shape. In some embodiments the frame 110 is attached to a supporting structure (not shown) such as a table, rack, cart, bed, bed mounted arm, or the like.

In certain embodiments, such as shown in FIG. 1, multiple cassette assemblies 120 are attached to the frame 110 to form a retractor 100. In some such embodiments the cassette assembly 120 is configured to be detachably attached to the frame 110 so that cassette assemblies 120 may be added or removed as needed for a particular application. A functional retractor may be configured using as few as two cassette assemblies 120 but the attaching of additional cassette assemblies can further increase functionality of the retractor in creating a surgical site.

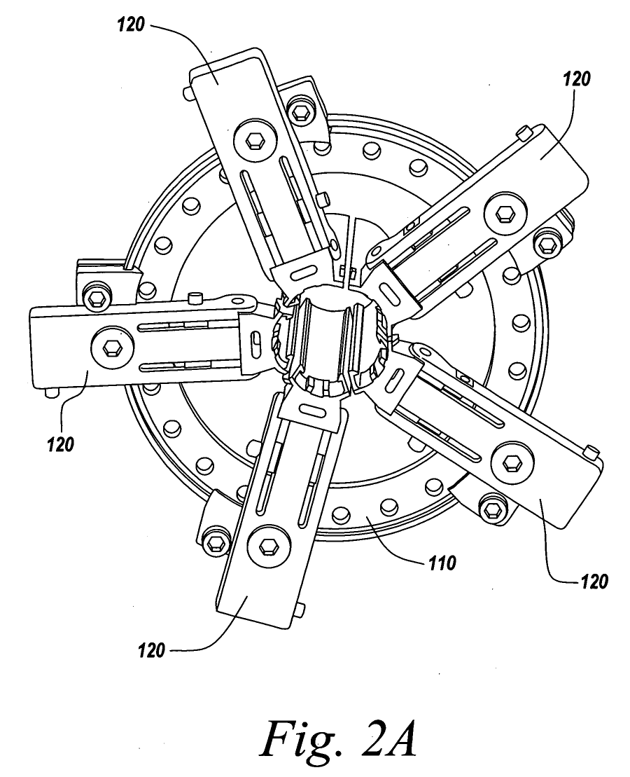

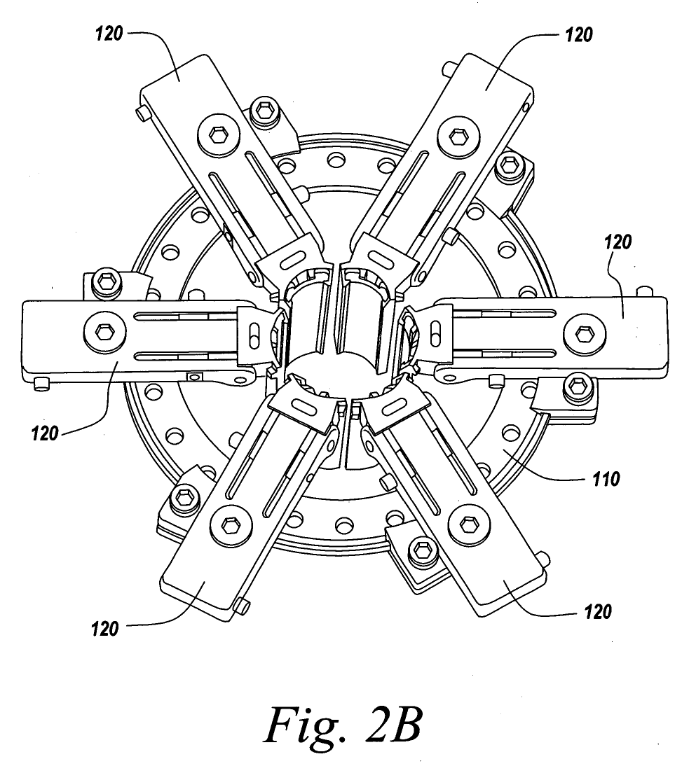

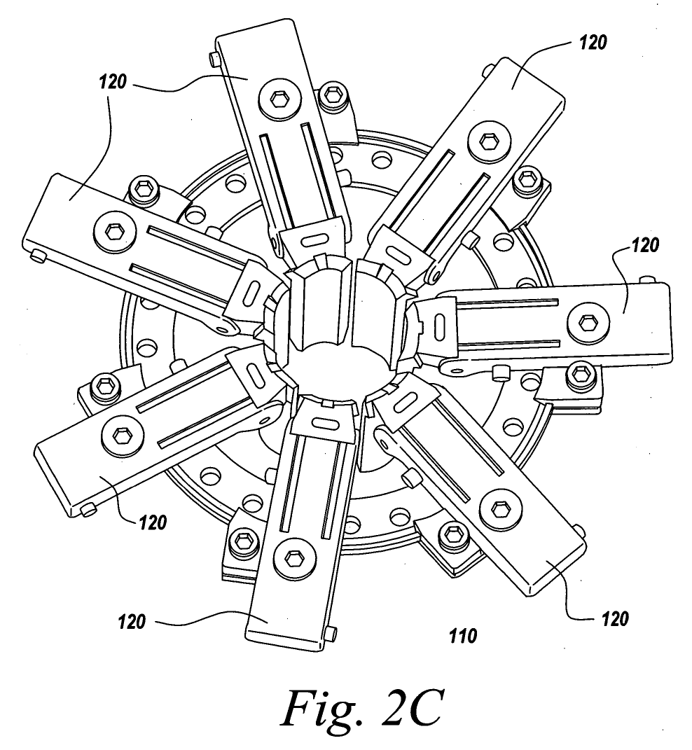

In the example of FIG. 1, four cassette assemblies 120 are attached to the frame 110 to form the retractor but still further combinations are possible as shown in FIGS. 2A-C. FIG. 2A depicts an embodiment having five cassette assemblies 120. FIG. 2B depicts an embodiment having six cassette assemblies 120. FIG. 2C depicts a seven cassette assembly embodiment.

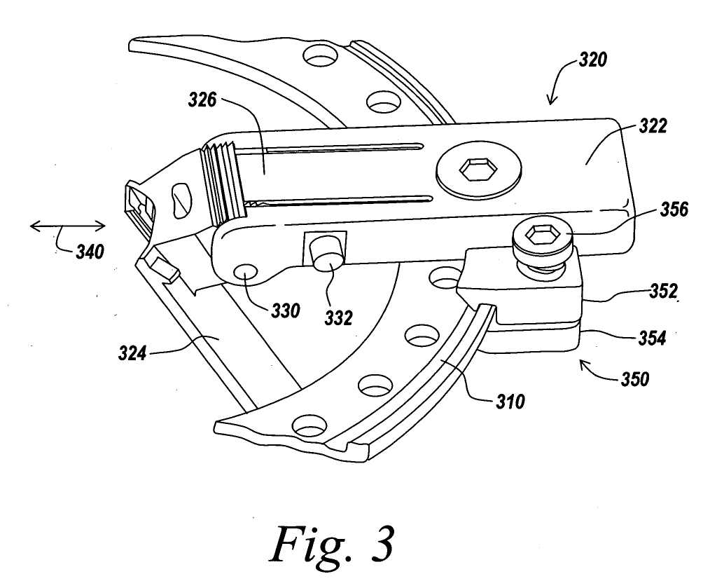

Referring now to FIG. 3, a close-up depiction of one embodiment of a cassette assembly 320 is shown. The cassette assembly 320 has a cassette housing 322, a blade 324, and first 326 and second 328 mechanisms. The cassette housing 322 is configured to be attached to the frame 310 of the retractor. The blade 324 is attached to the cassette housing 322 on a pivot 330. The first mechanism 326 is configured to control the pivot of the blade 324 so to allow a user to control and adjust angulation for the blade 324 when the retractor is in use. The second mechanism 328 is configured to control the retraction of the blade 324, in this case, allowing the user to control and adjust the radial movement of the blade depicted by arrow 340 on the frame 310. These elements will be discussed in more detail herein below.

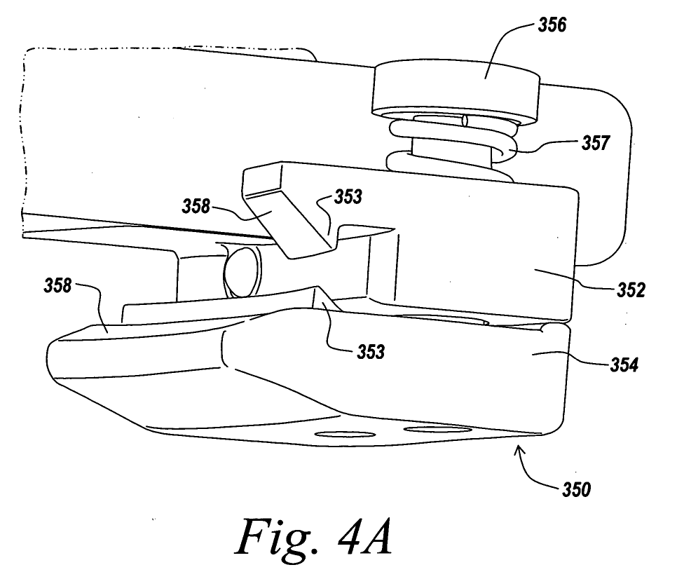

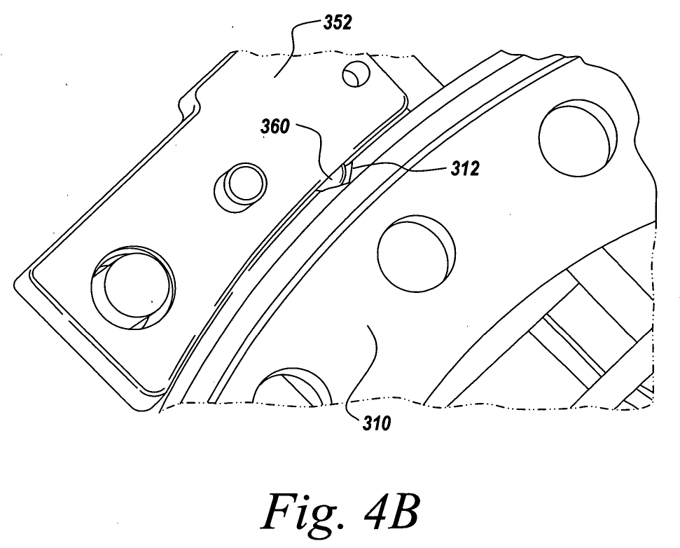

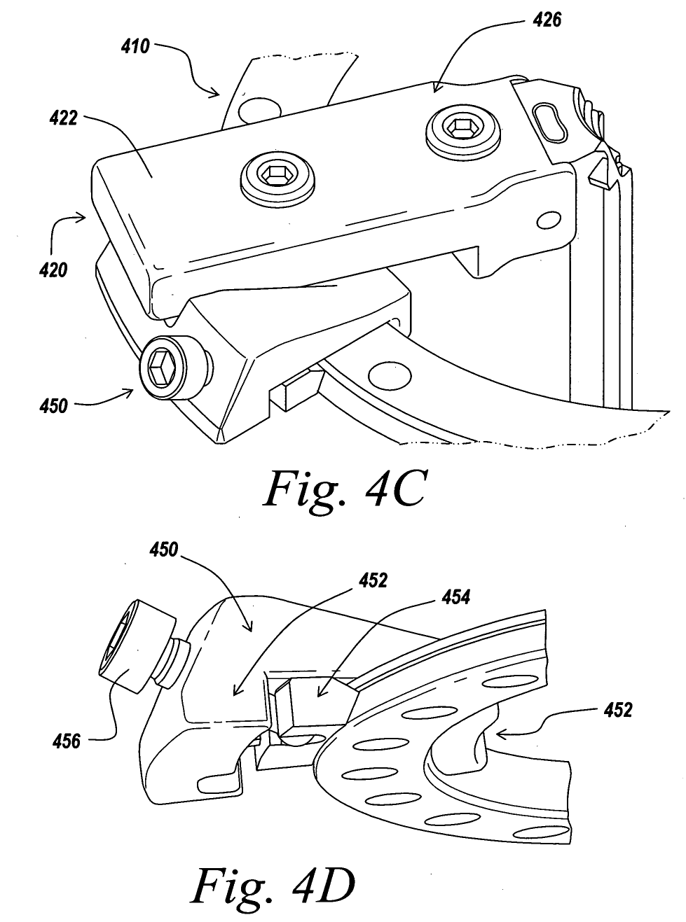

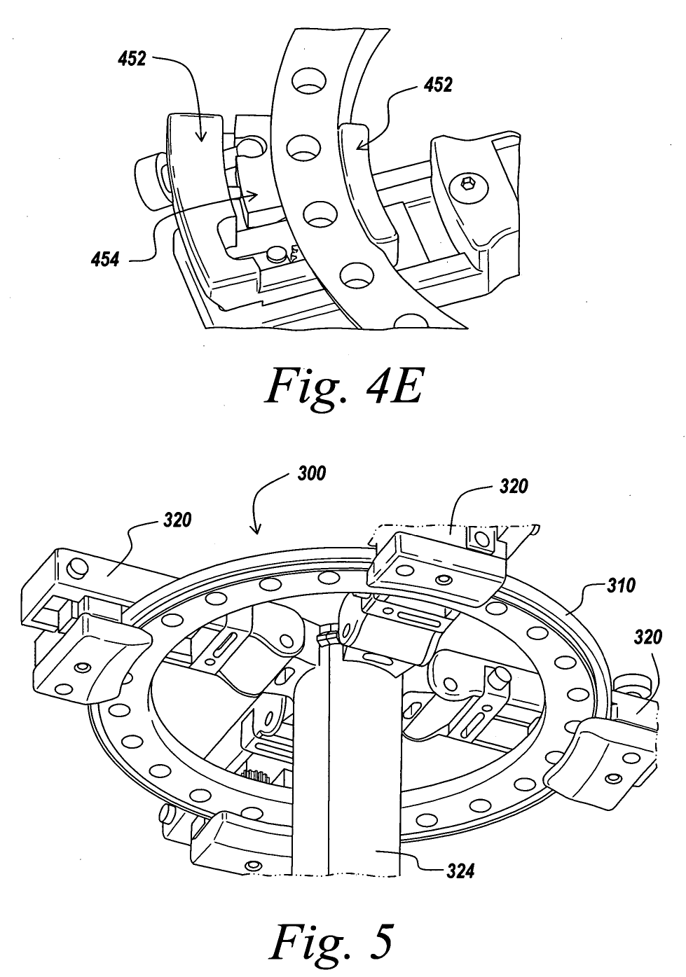

In the example embodiment of FIG. 3 the cassette housing 322 is attached to the frame 310 using a clamping mechanism 350. In this example the clamping mechanism 350 comprises an upper clamp portion 352, a lower clamp portion 354, and a locking mechanism 356 joining the upper clamp portion 352 to the lower clamp portion 354. The clamping mechanism is configured such that when the upper clamp portion 352 and the lower clamp portion 354 are joined or clamped together by the locking mechanism 356 the upper 352 and lower 354 portions engage the frame 310 securing the cassette housing 322 in place. A close-up depiction of this clamping mechanism 350 can be seen in FIG. 4A.

In the depiction of FIG. 4A the clamping mechanism 350 has been removed from the frame 310 and protrusions 353 on the upper 352 and lower 354 portions of the clamping mechanism 350 can be seen. The protrusions 353 are configured to mate with and engage the frame 310. The upper 352 and lower 354 portions also have a lead in feature 358 that allow easy insertion onto the frame. The locking mechanism in this embodiment is a screw 356 capturing a spring 357 which can be screwed down to clamp the upper 352 and lower 354 portions together. The spring 357 holds the upper 352 and lower 354 portions onto the frame 310 before the screw 356 is tightened locking the upper 352 and lower 354 portions together. Unscrewing the screw 356 releases the clamp and disengaging the clamping mechanism 350 from the frame 310. In some embodiments a positioning mechanism 360 such a ball-plunger that engages a notch on the frame 310 is provided. A depiction of how the positioning mechanism 360 operates can be seen in FIG. 4B.

FIG. 4B depicts an underside view of the upper portion 352 of the clamping mechanism 350 having a positioning mechanism 360. In this example the position mechanism engages a notch 312 on the frame 310. This provides a “home” position for the cartridge assembly 320 on the frame 310. The use of such “home” positions allow for quick assembly of a functional retractor.

Another embodiment for a clamping mechanism 450 can be seen in FIGS. 4C-E. In this embodiment the clamping mechanism is configured to allow the cassette assembly 420 to attach to the top of the frame or “top-load.” As with the previous embodiment the clamping mechanism 450 includes an upper portion 452, a lower portion 454 and a locking mechanism 456. In this embodiment, the upper portion 452 is configured to mount over the frame 410. The lower portion 454 is a wedge configured to be driven by the locking mechanism 456, here a screw, to engage the frame 410 against the upper portion 452 thereby securing the cassette housing 422 onto the frame 410. The advantage of such a top-loading mechanism 450 is that it allows for easy addition or removal of cassette assemblies 420 especially when the retractor has already been placed in-situ in the patient.

It should be noted that the preceding examples for clamping mechanisms are exemplary and but some of the embodiments possible. Other implementations and configurations will be apparent to one skilled in the art given the benefit of this disclosure.

Referring back to FIG. 3, the blade 324 is attached to a first end of the cassette housing 322 on a pivot 330. The blade 324 is configured such that when mated with one or more other blades on a retractor they form a tube for insertion into the body. An example of this can be seen in FIG. 5.

FIG. 5 is a depiction of the underside of a retractor 300 having four cassette assemblies 320 mounted on the frame 310. The blades 324 of the cassette assemblies 320 have been joined together in a “closed” position to from a tube. This tube can then be inserted into an incision and the blades can be retracted to open up a surgical site.

The length of the blade 324 is determined by the depth of the surgical site to be created. In certain embodiments the blade 324 is a telescoping blade such that the length of the blade can be adjusted as needed for a particular application. These are but some of the possible embodiments. Other implementations and configurations will be apparent to one skilled in the art given the benefit of this disclosure.

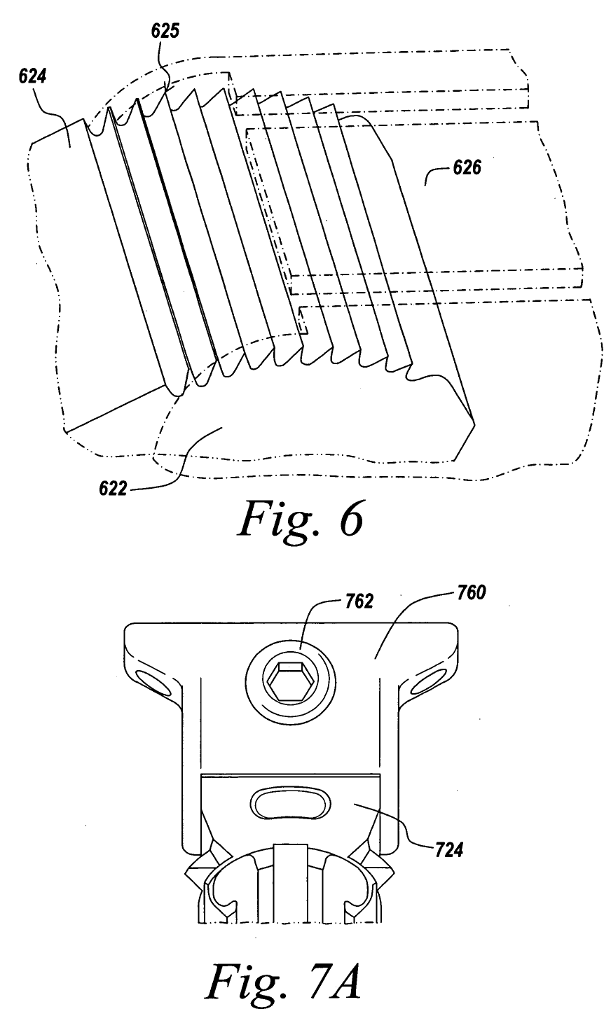

The rotation of the blade 324 around the pivot 330 is controlled by a first mechanism 326. In the embodiment of FIG. 3, the first mechanism 326 comprises a ratchet mechanism. In this example the ratchet mechanism 326 is a flexible tang formed as part of the housing 322. The flexible tang engages teeth on the blade 324. This engagement of the teeth can be seen in more detail in FIG. 6.

FIG. 6 depicts a close-up of the ratchet mechanism of FIG. 3. In this depiction the cassette housing 622 and flexible tang 626 are transparent to provide a view of the engagement of the teeth 625 on the blade 624. The teeth 625 and tang 626 are configured to allow the blade 624 to rotate in one direction but prevent rotation back in the opposite direction. That is, rotating the blade 624 around the pivot (not shown) causes ratchet with positive stop to occur holding the blade 624 in position.

In certain embodiments the first mechanism 326 may be provided with release 332 that disengages the blade 324 allowing the blade to freely rotate around the pivot 330. In the ratchet mechanism example of FIGS. 3 and 5A, the release 332 is a button that when pushed lifts up the tang 626 disengaging the tang 626 from the teeth 625 of the blade 624.

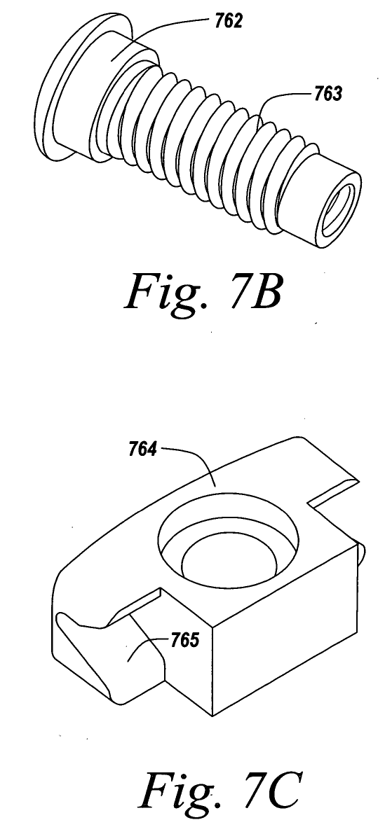

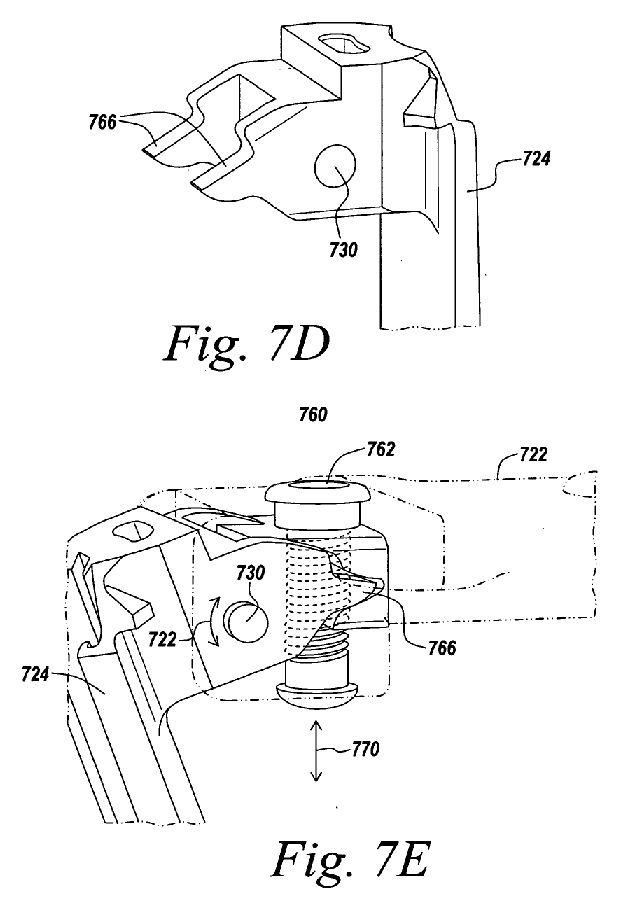

In another embodiment, as shown in FIG. 7A-E, the first mechanism comprises a vertical screw mechanism 760, formed as part of the housing 722, wherein the pivot of the blade 724 is controlled by a screw 762. The screw 762 is configured to be freely rotatable in the vertical screw mechanism 760. A nut 764 is configured to engage the threads 763 of the screw 762 and travel along the length of the screw 762 as the screw 762 is rotated. The nut 764 has one or more receiving openings 765. The blade 724 has protruding features 766 configured to engage the receiving openings 765 of the nut 764 such that when the nut 764 moves along the length of the screw 762, the blade 724 rotates around the pivot 730. The interaction of these elements can be seen in FIG. 7E. It should be understood however, that in certain embodiments, the elements may be reversed such that the blade has receiving features and the nut has protruding features.

The embodiment of FIG. 7E depicts a close-up of the vertical screw mechanism 760. In this depiction the cassette housing 722 is transparent to allow the interaction between the screw 762, nut 764, and blade 724 to be seen. As the screw 762 is turned, the threads 763 of the screw engage the nut 764 causing the nut 764 to move along the length of the screw 762 in the direction indicated by arrow 770. The receiving opening 765 of the nut 764 in turn engage the protrusion 766 of the blade 724 causing the blade 724 to rotate around the pivot 730 in the direction indicated by arrow 772. Thus, by rotating the screw 762 of the vertical screw mechanism 760 the pivot of the blade 724 is controlled.

Being able to control the pivot of the blade is important for the purpose of angling in the blades. That is, by rotating the blade 724 around the pivot 730 a user is able to sweep or push aside tissue inside a patient to create a surgical site without requiring additional retraction of the blade 724 Thus a surgical site can be created without requiring the incision opening to be increased.

It should be noted that the above described embodiments for controlling pivot of the blade are exemplary. Other possible implementation and configurations will be apparent to one skilled in the art given the benefit of this disclosure.

Referring again to FIG. 3, the second mechanism 328 controls the retraction of the blade 324. In this example retraction is performed by moving the blade of the cassette assembly 320 radially outward in the plane indicated by arrow 340. In the embodiment of FIG. 3, this radial movement is controlled by a rack and pinion mechanism. The rack and pinion mechanism can be seen in more detail in FIG. 8.

FIG. 8 depicts a close up of the rack and pinion mechanism 880. In this depiction the cassette housing 822 is transparent to provide view of the rack and pinion mechanism 880. In this embodiment, the rack and pinion mechanism 880 comprises a rotatable pinion 882 and a rack 884. The rotatable pinion 882 is attached to the cassette housing 822. The rack 884 is attached to a carrier 886 that is engaged with the cassette housing 822. The carrier moves in the cassette housing 822 in the direction indicated by arrow 890 while the rack 884 mates with the rotatable pinion 882. A pawl 888 may also be attached to the cassette housing 822 via a pivot 889 and engage the rotatable pinion 882. The pawl is spring loaded 892 to ratchet as the rotatable pinion 882 rotates and acts as a positive stop to prevent rotation in the opposite direction.

By fixing the position of the carrier 886 on the frame (not shown), rotating the pinion 882 causes the housing 822, and in turn the blade attached to the housing 822 (not shown) to move in the radial direction indicted by arrow 890. In certain embodiments, the rack and pinion mechanism 880 may also comprise a release, in this example, a push button, that disengages the pawl 888 allowing the rotatable pinion 882 to rotate freely.

It should be noted that the above described embodiment for controlling retraction of the blade is exemplary. Other possible implementation and configurations will be apparent to one skilled in the art given the benefit of this disclosure.

The retractors and cassette assemblies of the present invention can be made of those materials that are commonly used in medical devices. Examples of suitable materials include, but are not limited to, metals and metal alloys (e.g., stainless steel, aluminum, titanium, nitinol, cobalt chrome, etc.), plastics (e.g., carbon fiber reinforced polyethylene (CFRP), ultra high molecular weight polyethylene (UHMWP), ultem, radel, vectra, polycarbonate, etc.). In some embodiments of the invention, the retractor includes a radiolucent material (e.g., radiolucent plastics, aluminum, thin stainless steel, titanium, nitinol, or cobalt chrome). In further embodiments of the invention, the retractor and cassette assemblies include radio-opaque materials. In still other embodiments, an illuminant may be coupled with the retractor or cassette assemblies.

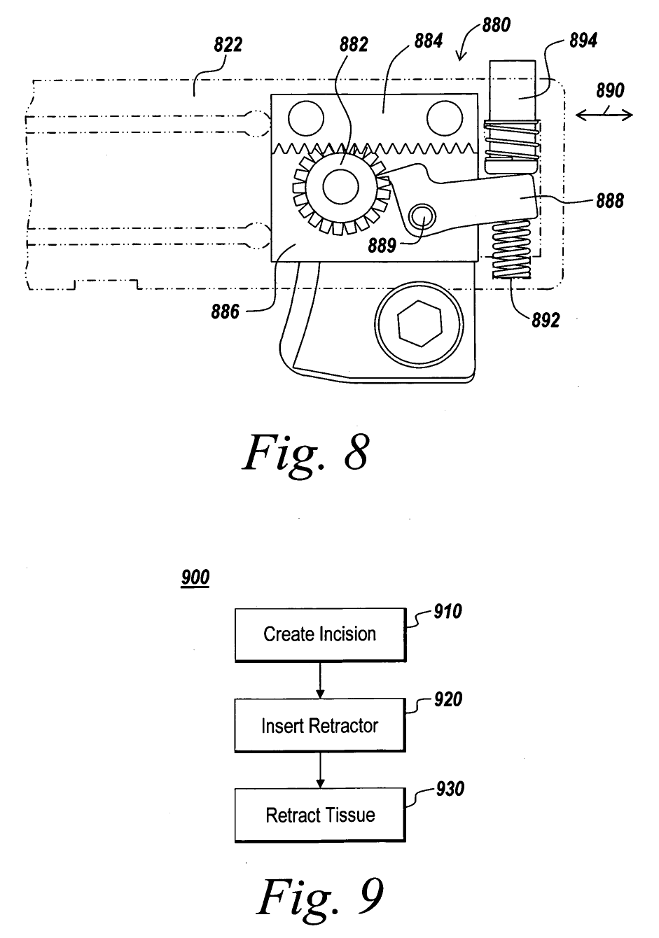

FIG. 9, depicts a flow chart 900 for one exemplary embodiment of a method for forming a surgical site in a patient. The method involves the steps of creating and incision in the patient 910, inserting a retractor into the incision 920, and retraction the tissue of the patient at the incision with the retractor. These steps are discussed in more detail below.

In some embodiments, the method includes making a first incision in the epidermis of the patient and then expanding the incision into a portion of the subdermal tissue to create a pathway in any conventional manner. For example, the incision can be expanded by dilation to the desired shape, and orientation by using a plurality of dilators. Once the incision has been expanded to the desire size, shape, and orientation the retractor may be inserted.

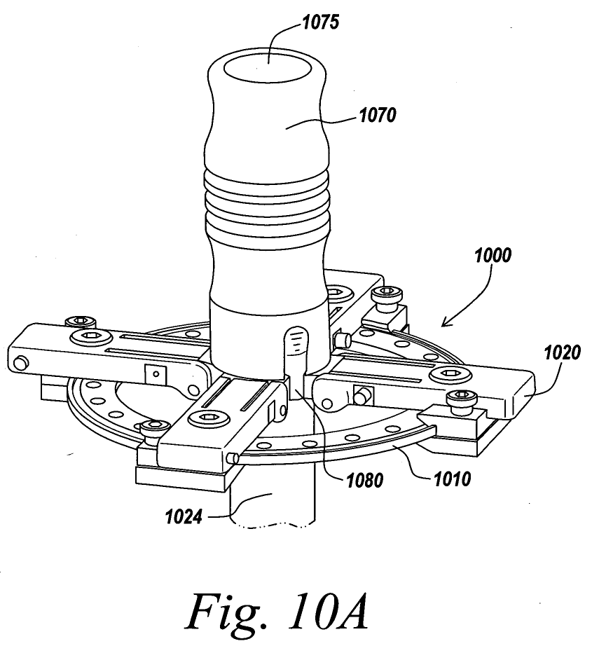

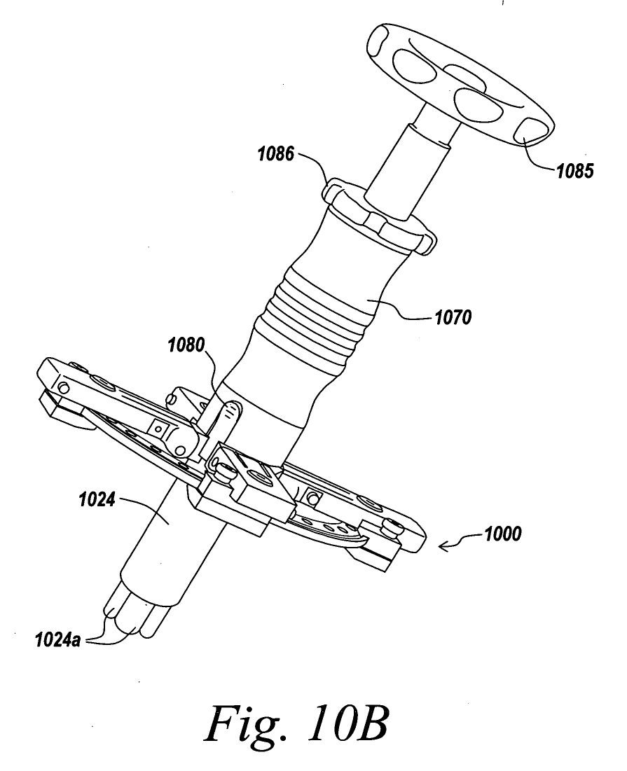

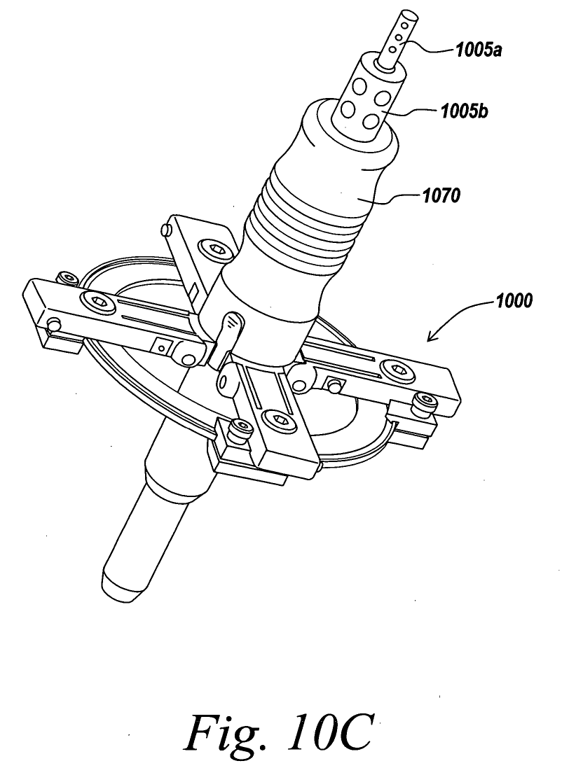

In certain embodiments an insertion instrument is used to insert the retractor. An example of such an insertion instrument 1070 can be seen in FIG. 10A. FIG. 10A depicts an insertion instrument 1070 that comprises a handle. The handle has fasteners 1080 that attach the handle to the blades 1024 of the retractor 1000. In certain embodiments, the handle 1070 may also have a central bore 1075 allowing other instruments to pass through the handle 1070. An example of this can be seen in FIGS. 10B and 10C.

In the embodiment of FIG. 10B, the blades 1024 of the retractor 1000 have a telescoping feature 1024a. The length of the telescoping blades 1024a is set using a blade depth adjustment tool 1085. The blade depth adjustment tool 1085 is inserted through the handle 1070 and engages the telescoping blades 1024a extending the telescoping blades 1024a. The amount the telescoping blades are extended is determined by an adjustable stop 1086 on the blade depth adjustment tool 1085. Exemplary tissue engaging blades having an adjustable length, e.g., telescoping blades, are disclosed in U.S. Patent Application Publication No. 2005-0137461 A1, which is incorporated herein by reference.

In the embodiment of FIG. 10C, serial dilation is used to prepare the incision for the insertion of the retractor 1000. As such a series of dilators 1005a, 1005b may be inserted into the patient through the incision (not shown). The retractor 1000 may then be inserted into the insertion over the dilators 1005a, 1005b using the insertion device 1070. The dilatators 1005a, 1005b passing though the retractor and insertion instrument 1070 serve as a guide for the insertion of the retractor 1000.

It should be understood that the above embodiments are exemplary. Other possible insertion techniques with or without insertion instruments as well as different insertion instruments are possible. Other implementations and configurations will be apparent to one skilled in the art given the benefit of this disclosure.

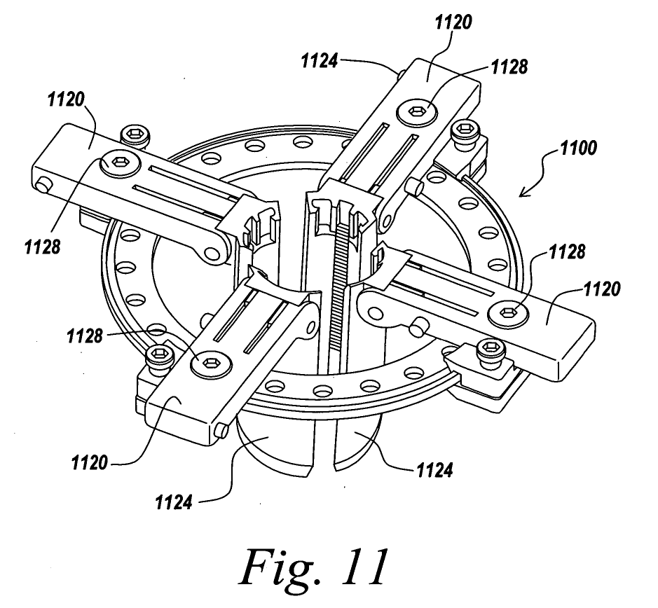

Once the retractor has been inserted, the tissue of the patient can be retracted by retracting the blades of the retractor. An example of this can be seen in FIG. 11. Here the retractor is opened up by retracting the blades 1124 of the cassette assemblies 1120 using the second mechanism 1128 of the cassette assemblies 1120. In this embodiment the second mechanism 1128 is a rack and pinion mechanism that controls the retraction of the blades 1124. By turning the pinions 1028 the cassette housings, and attached blades 1124 is moved radially outward. This in turn retracts the tissue of the patient creating a surgical site. If the blades 1124 are retracted too far, a release 1184 may be used to disengage the rack and pinion. The natural elasticity of the tissue will push the blade 1124 back.

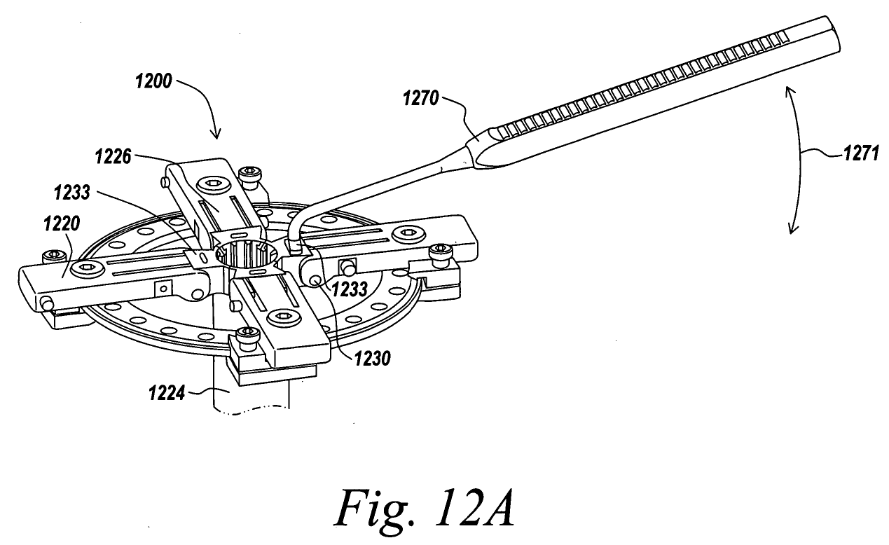

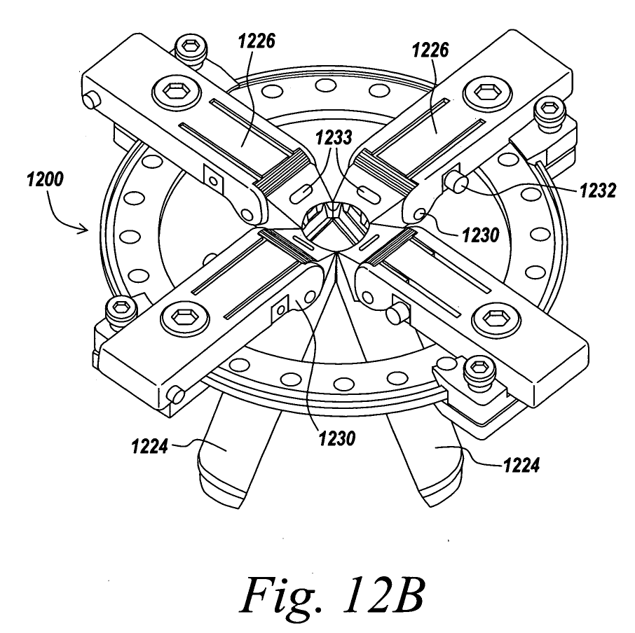

In addition to the retraction of the blades, the angulation of the blades may also be set. An example of this can be seen in FIGS. 12A and 12B. In FIG. 12A, an angulation adjustment instrument 1270 is used to set the angulation for the blades 1224. The angulation adjustment instrument 1270 is configured to engage a notch 1233 in the blade 1224. Once the notch 1233 is engaged by the angulstion adjustment instrument 1070, moving the instrument in the direction indicated by arrow 1271 causes the blade 1224 to rotate around the pivot 1230. The ratchet of the first mechanism 1226 provides a positive stop maintaining the position of the blade 1224. An example of a retractor 1200 with the blades 1224 toed in can be seen in FIG. 12B.

In the embodiment of FIG. 12B the blades 1224 of the retractor 1200 have been toed-in. That is, the blades 1224 have been rotated around the pivot 1230 to a desired position. The ratchet of the first mechanism 1226 maintains the position of the blades 1224. If one of the blades 1224 is over rotated the release 1232 may be used to disengage the first mechanism 1226. The natural elasticity of the tissue of the patient will then push the blade back removing the toe-in. Using the angulation feature of the retractor 1200 allows for the clearing of tissue from a surgical site without requiring the size of the incision to be increased as the blades 1224 of the retractor 1200 hold the tissue away from the surgical site underneath the surface where the incision was made.

The apparatus and techniques of the present invention provide numerous advantages. Using the retractor of the present invention can be used in any approach, including lateral, posterior, and anterior. The retractor is highly adaptive, in that additional cassette assemblies can be used for increased retraction.

Although, the present invention has been described relative to an illustrative embodiment and application in spinal correction surgery. It should be apparent that the present invention may be used in any number of surgical procedures. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

What is claimed is:

1. A cassette assembly for use in a surgical retractor, the cassette assembly comprising:
a cassette housing configured to attach to a frame;
a blade pivotably connected to the cassette housing;
a first mechanism configured to control the pivot of the blade; and
a second mechanism configured to control retraction of the blade.

2. The cassette assembly of claim 1 wherein the cassette housing is configured to be top loaded onto a frame.

3. The cassette assembly of claim 1 wherein the cassette housing is configured to be detachably attached to a frame.

4. The cassette assembly of claim 1 wherein the first mechanism comprises a ratchet mechanism that engages the blade and controls the pivot of the blade.

5. The cassette assembly of claim 4 wherein the ratchet mechanism further comprises a release that disengages the blade allowing the blade to pivot freely.

6. The cassette assembly of claim 1 wherein the first mechanism comprises a vertical screw mechanism that engages the blade and controls the pivot of the blade.

7. The cassette assembly of claim 1 wherein the second mechanism comprises a rack and pinion mechanism that controls the retraction of the blade.

8. The cassette assembly of claim 7 wherein the rack and pinion mechanism further comprises a release mechanism that disengages the rack and pinion.

9. The cassette assembly of claim 1 wherein the blade comprises a telescoping blade.

10. A surgical retractor comprising:
a frame; and
a cassette assembly attached to the frame, the cassette assembly including
a cassette housing configured to attach to the frame;
a blade pivotably connected to the cassette housing;
a first mechanism configured to control the pivot of the blade; and
a second mechanism configured to control retraction of the blade.

11. The surgical retractor of claim 10 having a plurality of cassette assemblies attached to the frame.

12. The surgical retractor of claim 10 wherein the cassette assembly attaches to the top of the frame.

13. The surgical retractor of claim 10 wherein the cassette assembly is detachably attached to a frame.

14. The surgical retractor of claim 10 wherein the frame is substantially circular.

15. The surgical retractor of claim 10 wherein the first mechanism of the cassette assembly comprises a ratchet mechanism that engages the blade and controls the pivot of the blade.

16. The surgical retractor of claim 15 wherein the ratchet mechanism further comprises a release that disengages the blade allowing the blade to pivot freely.

17. The surgical retractor of claim 10 wherein the first mechanism of the cassette assembly comprises a vertical screw mechanism that engages the blade and controls the pivot of the blade.

18. The surgical retractor of claim 10 wherein the second mechanism of the cassette assembly comprises a rack and pinion mechanism that controls the retraction of the blade.

19. The surgical retractor of claim 18 wherein the rack and pinion mechanism further comprises a release mechanism that disengages the rack and pinion.

20. The surgical retractor of claim 10 wherein the blade of the cassette assembly comprises a telescoping blade.

21. A method of forming a surgical site in a patient, the method comprising:
creating an incision in the patient;
inserting a retractor into the incision in the patient, the retractor including
a frame; and
a cassette assembly attached to the frame, the cassette assembly including
a cassette housing configured to attach to a frame;
a blade pivotably connected to the cassette housing;
a first mechanism configured to control the pivot of the blade; and
a second mechanism configured to control retraction of the blade;
retracting the tissue of the patient at the incision with the retractor to form a surgical site.

22. The method of claim 21, wherein the surgical site provides access to the spine of the patient.

23. The method of claim 21, wherein a dilator is inserted through the incision and retractor is inserted over the dilator.

24. The method of claim 21, wherein retractor is inserted into the incision using an insertion instrument.

25. The method of claim 21, wherein the step of retracting the tissue of the patient further comprises adjusting the retraction of the blade using the second mechanism.

26. The method of claim 21, wherein the step of retracting the tissue of the patient further comprises adjusting the pivot of the blade using the first mechanism.
